(19) United States
(12) Patent Application Publication
(10) Pub. No.: US 2017/0319850 A1
(43) Pub. Date: Nov. 9, 2017

(54) TREATMENT METHOD INCLUDING THE USE OF ENCODING BIO-RESONANT ENERGY WAVES

(71) Applicant: Hans Thomas Meyer, Richland, WA (US)

(72) Inventor: Hans Thomas Meyer, Richland, WA (US)

(21) Appl. No.: 15/587,651

(22) Filed: May 5, 2017

Related U.S. Application Data
(60) Provisional application No. 62/333,269, filed on May 8, 2016.

(51) Int. Cl. A61N 1/36; H04L 29/08; A61N 1/04

(52) U.S. Cl. CPC A61N 1/36014; A61N 1/0472; H04L 67/06

(57) ABSTRACT

A method of providing therapeutic regimens includes encoding a plurality of treatment regimens each in a treatment digital file, a treatment regimen of the plurality including a plurality of simultaneous frequency signals; and distributing the plurality of treatment regimens to a file marketplace. A method for treating a patient includes encoding a therapeutic frequency in a file to be stored on a device having an audio jack with sufficient capabilities to deliver the specific treatment, applying electrodes connected to the device through the audio jack to the patient, and applying the treatment with the device. The therapeutic frequencies are applied to the patient as electrical energy waves.

### CROSS-REFERENCE TO RELATED APPLICATION(S)

This application claims benefit of U.S. Provisional Application No. 62/333,269, filed May 8, 2016, which is incorporated herein by reference in its entirety.

### FIELD OF THE DISCLOSURE

This disclosure, in general, relates to a treatment system and methods for treating tissues of a patient.

### BACKGROUND

Increasingly, the public is becoming concerned about the side effects of pharmaceutical intervention for common ailments. For example, the side effects of pharmaceutical treatment of acne can be more detrimental than the problem to be treated. For acne, for example, the side effects of pharmaceutical treatments include sensitivity to sunlight, peeling skin, rash, itching, abdominal pain, nausea, vomiting, diarrhea, constipation, inflammation of the esophagus, swollen tongue, joint pain, and ringing in the ears.

In other examples, there are few treatment options for some conditions, and patients are told to wait to determine whether the condition will improve. For example, Bell's palsy is a condition for which effective treatments are limited. While corticosteroids have been shown to positively affect 14% of patients, patients are often advised to wait to see whether the condition improves over time.

In addition, many treatment options are in the form of pills or ointments that carry with them the inconvenience of acquiring the treatment option, finding time and place to apply or ingest, and the associated mess, bitter taste, or unpleasant smells.

As such, improved treatment options would be desirable.

### SUMMARY

In a first aspect, a method of providing therapeutic regiments includes encoding a plurality of treatment regimens each in a treatment digital file, a treatment regimen of the plurality including a plurality of simultaneous frequency signals; and distributing the plurality of treatment regimens to a file marketplace.

In a second aspect, a method of treating a patient includes selecting a treatment regimen from a plurality of treatment regimens available through a user interface of a device, the device including an analog output and the user interface, each treatment regimen of the plurality of treatment regimens associated with a file encoding a digital treatment signal; decoding the file using the device to produce the digital treatment signal; converting the digital signal to an analog treatment signal; and applying the analog treatment signal as an electric signal through the analog output and to a patient using an electrode in electrical communication with the analog output and the patient.

In a third aspect, a method for treating a patient includes encoding a therapeutic frequency in a digital file to be stored on a device having an audio jack; applying electrodes connected to the device through the audio jack to the patient; and playing the digital file with the device, the therapeutic frequencies applied to the patient as electrical energy waves.

In a fourth aspect, a method of treating a patient includes decoding a digital file that has been encoded with a set of therapeutic frequencies using an audio codec of a device, the device including an audio jack; applying electrodes to a patient, the electrodes electrically connected to the audio jack of the device; and transmitting an electrical energy wave signal corresponding to the therapeutic frequencies through the patient via the electrodes.

In a fifth aspect, an apparatus includes a processor; a memory storage in communication with the processor; a power supply; an interface in communication with the processor; and a digital-to-analog converter in communication with the processor and a jack having an audio configuration; and a set of electrodes in electrical communication with the jack; wherein the processor is to retrieve an encoded treatment regimen from the memory storage in response to selection of the treatment regimen via the interface, decode the treatment regimen to a digital treatment signal, convert the digital treatment signal to an analog treatment signal using the digital-to-analog converter, and apply the a analog treatment signal as electrical energy waves to the electrodes.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of an exemplary treatment system.

FIG. 2 includes an illustration of an exemplary treatment device.

FIG. 3 includes an illustration of an exemplary circuitry.

FIG. 4 includes an illustration of an exemplary portable treatment device.

FIG. 5 and FIG. 6 include illustrations of exemplary signals.

FIG. 7 and FIG. 8 include illustrations of exemplary circuitry.

FIG. 9 includes an illustration of an exemplary clip contact.

FIG. 10 includes an illustration of an exemplary pad contact.

FIG. 11 includes a flow diagram of an exemplary method.

The use of the same reference symbols in different drawings indicates similar or identical items.

### DETAILED DESCRIPTION

In an exemplary embodiment, a system for delivering a treatment to a patient or user includes providing an audio format file encoding a treatment regimen to a user device. For example, the audio format file can be downloaded from an audio library and stored on a user device. In another example, the audio format file can be streamed to the user device through a network. The user device can decode the audio file into a treatment signal and can apply the treatment signal to the user or patient as electrical energy waves. In an example, the user device can be a mobile device, such as a smart phone or tablet, capable of decoding the audio format file and having an analog audio jack.

In another exemplary embodiment, a device provides an electrical energy waves across a tissue of a patient or user. In an example, the electrical signal includes a combination of multiple therapeutic frequencies. In a particular example, the device is a mobile device having an analog output, such as an audio jack. A set of leads connected to electrodes are electrically connected or coupled with the analog output. The electrodes are in contact, such as in direct contact or capacitively coupled, with the tissue of the patient. In an example, the tissue includes the skin. In an example, the electrical signal includes a combination of two or more therapeutic frequencies. Exemplary frequencies are in a range of 0.1 Hz to 2000 kHz.

In a particular example, the therapeutic frequencies are encoded in a digital file that can be decoded using an audio codec that incorporates the therapeutic frequencies. The digital signal can be converted using a digital-to-analog converter and applied to the patient as an analog electric energy wave signal incorporating the therapeutic frequencies.

In a further exemplary embodiment, a method includes encoding therapeutic frequencies in an audio type format. Using a device, such as an audio player, mobile device, smart phone or tablet, a user can select a treatment regimen associated with an audio file encoding the therapeutic frequencies. The digital file can be decoded using an audio codec to recover the therapeutic frequencies and played or delivered through an analog output, such as the audio output, of the device. The user or patient can attach electrodes electrically coupled to the analog output of the mobile device to the skin. For example, the patient or user can apply the electrodes to the ears of the patient. In another example, the electrodes can include pads to contact the skin. In an alternative example, the electrodes can disrupt the skin, for example, using needles.

Energy wave frequencies are mapped into a frequency domain spectrum then converted to a time domain spectrum. Energy wave frequencies can be chosen based on the resonant frequencies of the particular ailment.

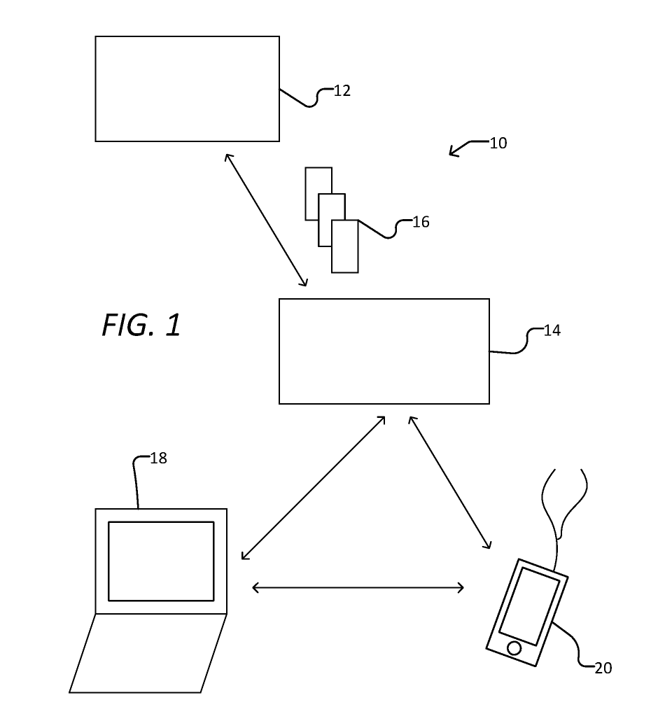

In an example, FIG. 1 includes an illustration of an exemplary system 10 for delivering a treatment to a user or patient. In an example, a treatment regimen can be encoded in a set of audio format files 16 using a computational device 12. The audio format files 16 can encode one or more treatment regimens. Exemplary audio formats include a format compliant with a Moving Picture Experts Group (MPEG) standard (e.g., MPEG-1, MPEG-2, MPEG-4, or combinations thereof), AAC, ALAC, WAV, WMAL, a custom format, or the like, or a combination thereof. In particular, the therapeutic regimen can include therapeutic frequencies to be applied to a patient or user as electrical energy waves, for example, having a frequency in a range of 0.1 Hz to 2000 kHz.

The audio format files 16 can be supplied to a networked server 14. In an example, the networked server 14 provides an audio library or file sharing capabilities over a network. For example, the networked server can provide a website through which the audio format files 16 are accessible. In another example, the networked server 14 can implement an audio library through which a user can purchase the audio format files 16, among other audio format files. In another example, the networked server 14 can deliver streaming content incorporating the audio format files 16.

In an example, a computational device 18, such as a laptop or desktop computer, can access the networked server 14 to acquire the audio format files 16. For example, the computational device 18 can access the networked server 14 through a global network, such as the Internet. The computational device 18 can have an analog output, such as an analog audio jack. A user can receive treatment by decoding the audio format file on the computational device 18 and applying the treatment regimen to the user through electrodes connected to the analog output.

In another example, a user device 20, such as a mobile audio player, smart phone, or tablet, can access the audio format files 16 stored on the computational device 18. The user device 20 can have an analog output, such as an analog audio jack. The user can receive treatment by decoding the audio format file on the user device 20 and applying the treatment regimen through electrodes connected to the analog output of the user device 20. In an alternative example, the user device 18 can access the networked server 14 directly, for example, through a global network. The networked server 14 can provide the audio format files 16 to the user device 20 or can stream the content of the audio format files 16 to the user device 20.

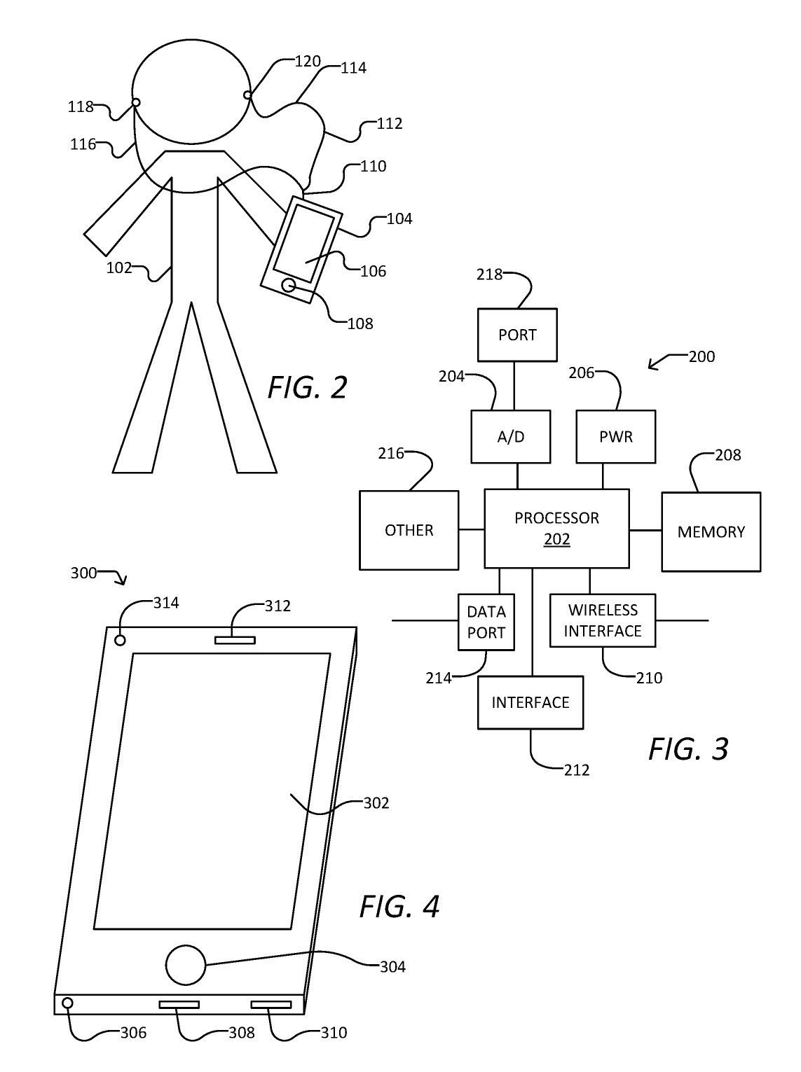

In an example illustrated in FIG. 2, a patient or user 102 can access a device 104 that applies a treatment regimen comprising a set of therapeutic frequencies to the user or patient 102. The device 104 can include a visual interface 106, such as a touchscreen, and optionally one or more interfaces 108, such as buttons or switches. The device 104 can further include a port 110, such as an analog port, for example an audio jack. The port 110 can be connected by leads 112 to clips or contact pads 114 or 116, which attach at regions of the skin 118 or 120, such as the ears or fingers of the patient 102. The device 104 can deliver through the port 110 to the user or patient 102 an analog electrical signal incorporating the therapeutic frequencies. In an example, the device is an audio player, mobile player, smart phone, tablet, or laptop.

As illustrated in FIG. 3, a circuitry 200 incorporated into a device, such as the device 104 illustrated in FIG. 2, can include a processor 202. The processor 202 can be coupled to a power storage 206 from which it draws power. In addition, the processor 202 can be communicatively coupled to a memory 208. The memory 208 can store in non-transitory forms software operable by the processor 202 to implement various methods described herein. In particular, the memory 208 can store one or more digital files, such as audio files, encoding combinations of therapeutic frequencies.

Further, the processor 202 can be coupled to a user interface 212. In particular, the interface 212 can include one or more buttons, displays, or touchscreens. In an example, the user interface 212 can include a touchscreen, button, stylus, microphones, speakers, printer devices, or a combination thereof. In a particular example, the user interface 212 includes volume controls. The user can control the intensity of a treatment being applied by adjusting the volume controls.

The user can access the interfaces 212 to select a file including a treatment regimen stored on memory 208. The processor 202 can decode the file. For example, the processor 202 can decoded an audio file encoding the therapeutic frequencies using an audio codec and can apply the digital signal generated by decoding the audio file to the digital-to-analog converter 204, which is in electrically communication with the processor 202. In an example, the digital-to-analog converter 204 is coupled to a port 218, such as an audio jack. Exemplary audio jacks include 2.5 mm audio jacks, 3.5 mm audio jacks, 6.35 mm audio jacks, or the like. Optionally, the audio jack can be configured to receive an input, for example, from a microphone, in addition to providing an output. In such a configuration, the audio jack can be used to receive a measurement signal from a sensor, and the device 200 can convert the measurement signal to a digital signal to be processed by the processor 202.

Further, the processor 202 can be in electrical communication with other devices or circuitry 216. For example, the other devices or circuitry can include cameras, lights, mechanism for implementing vibration modes or audio signals, among others, or any combination thereof. In addition, the processor can be in communication with one or more wireless interfaces 210. For example, the wireless interface 210 can include a cellular or mobile phone interface, a mobile data interface, a Bluetooth™ interface, a Wi-Fi interface, or any combination thereof. In particular, the wireless interface 210 can be used to download data or stream audio files. The device 200 can also include a power input 214 to provide power to the power storage 206.

For example, FIG. 4 illustrates an exemplary device 300. The device 300 can be a mobile device, such as a mobile phone or tablet device. The device 300 includes a set of user interfaces, such as a display 302, one or more buttons 304, a microphone 310, or a speaker 312. In an example, the display 302 can be a touchscreen display. The device 300 can also include other circuitry, such as a camera 314. In addition, the device 300 can include a data interface or a power input, such as a combination port 308 (e.g., a USB port) configured to receive data or power.

Further, the device 300 can include an analog port 306. In an example, the analog port 306 is an audio jack, such as a 2.5 mm audio jack, 3.5 mm audio jack or 6.35 mm audio jack. Leads to be electrically coupled to a patient can be connected to the device 300 through the analog port 306. In general, one or more leads are connected to the patient from the analog port 306 of the device 300.

In particular, a user can download to the device 300 one or more treatment regimens stored or encoded in a file, such as an audio format. Exemplary audio formats include a format compliant with a Moving Picture Experts Group (MPEG) standard (e.g., MPEG-1, MPEG-2, MPEG-4, or combinations thereof), AAC, ALAC, WAV, WMAL, a custom format, or the like, or a combination thereof. A user can select using the user interfaces 302-304 or data input 308 a desired treatment regimen having an associated audio file. The device 300 can decode the treatment regimen from the file, for example, using an audio codec, to provide a digital signal encoding therapeutic frequencies. The device 300 can include a digital-to-analog converter to convert the digital signal to an analog electric signal incorporating the therapeutic frequencies, which are provided through the analog port 306, such as an audio analog jack.

In particular, therapeutic frequencies are applied to the user or patient in the form of electrical pulses or signals. The therapeutic frequencies can have a range of 0.1 Hz to 2000 kHz. For example, the therapeutic frequencies can have a range of 0.1 Hz to 1000 kHz, such as a range of 70 Hz to 10 kHz, a range of 80 Hz to 1 kHz, a range of 80 Hz to 500 Hz, or a range of 100 Hz to 200 Hz.

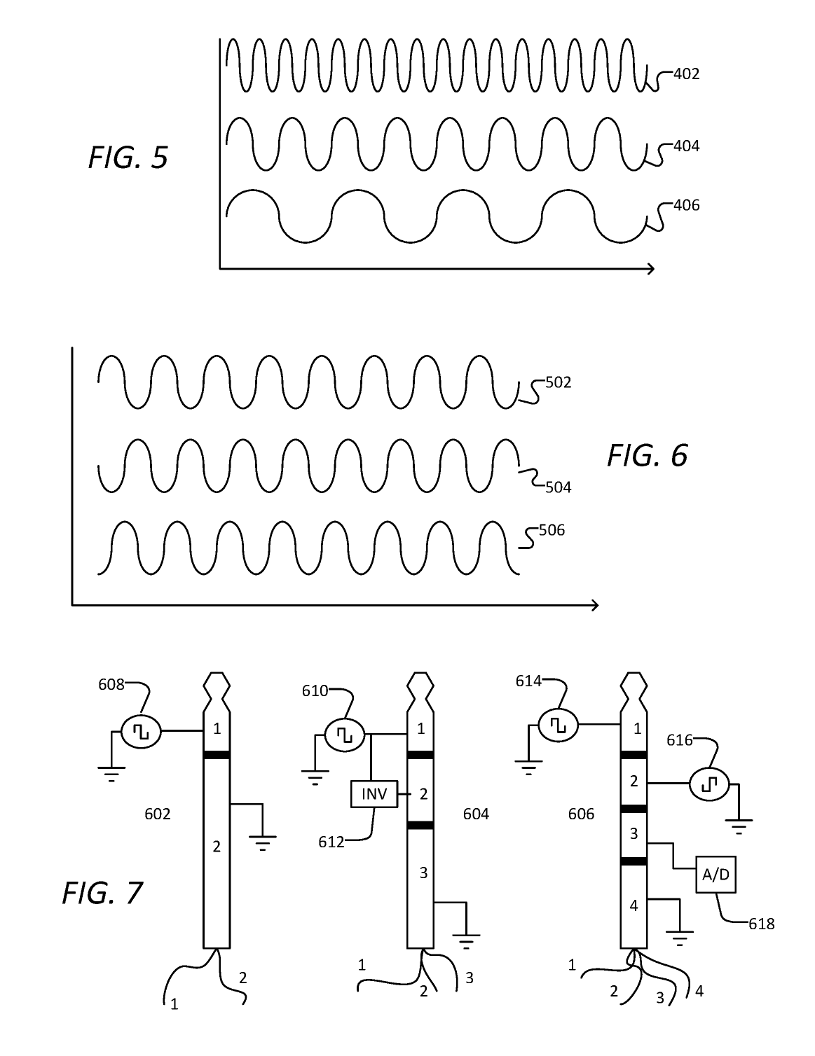

In particular, the electrical signals applied to the patient can be a combination of therapeutic frequencies. For example, as illustrated in FIG. 5, two or more therapeutic frequencies of different frequency ranges can be applied in combination to a patient. For example, the therapeutic frequency 402 can be added to the therapeutic frequency 404 and applied to the patient concurrently, such as simultaneously. In another example, therapeutic frequencies 402, 404, and 406 can be applied concurrently to the patient within the analog electric signal. In another example, a set of therapeutic frequencies can be applied sequentially. For example, the first set of two or more therapeutic frequencies can be applied simultaneously to a patient followed by a second set of therapeutic frequencies. In an example, the therapeutic frequencies can be applied for duration of 15 seconds to 30 min. For example, the duration can be between 15 seconds and 20 minutes, such as between 30 seconds and 15 minutes, 1 minute and 15 minutes, 1 minute to 10 minutes, or 1 minute to 8 minutes. The treatment regimen can be applied between 1 and 10 times per day, such as between 3 and 8 times.

The therapeutic frequencies can be applied to the patient through one or more leads. In the case of a single channel lead, the patient is assumed to be electrically grounded. As such, a single lead can be applied to the patient and a single channel can be used to provide the therapeutic frequencies to patient. In another example, at least two leads can be applied to patient; the first lead can include the therapeutic frequencies and, the second lead can be electrically coupled to ground.

In an alternative example, a two-channel system, such as a stereo output from an audio jack, can be utilized to carry the therapeutic frequencies. Leads associated with each channel can be attached or electrically coupled to the patient, and optionally a ground can be coupled to the patient. In such an example, the first channel can carry a first set of therapeutic frequencies, and the second channel can carry a second set of therapeutic frequencies. As illustrated in FIG. 6, the first set of therapeutic frequencies 502 can be an inverse of the second set of frequencies 504. In an alternative example, the second set of therapeutic frequencies (e.g., 506) can have a different start time than the first set of therapeutic frequencies 502.

Leads configured to carry the signal to the patient can interface with the device using various formats. For example, as illustrated in FIG. 7, a lead 602 can be configured to carry a single channel. For example, the first contact (1) of the lead 602 when inserted into the device can electrically couple with a signal generator 608 and a second contact (2) when inserted into the device can be electrically coupled to ground. The two contacts (1 and 2) can be electrically connected to the patient using clips or pads to apply either direct electrical contact or capacitively couple the leads to patient.

In another example, a two-channel lead 604 can be connected to the audio device. The first contact (1) when connected to the audio device can be electrically coupled to a signal generator 610. A second contact (2), for example a stereo channel, can be coupled also to signal generator 610 and optionally, can be coupled through an inverter 612. In such an example, the second contact (2) carries an inverted signal of the signal applied to the first contact (1). In an alternative example, the second contact (2) can be coupled to a different signal generator that provides a separate signal to the second contact (2). Alternatively, the contacts (1 and 2) can be coupled to the same signal generator, which supplies a stereo signal based on the therapeutic frequencies encoded into an audio file. The audio file can be encoded with stereo frequencies that are inverted from one another or are distinct sets of therapeutic frequencies. A third contact (3) can be coupled to ground, and optionally connected to the patient.

In another example, a lead 606 can include an additional channel, such as a channel traditionally used for a microphone. Stereo channels (1 and 2) can be coupled to a signal generator 614 or 616. A third channel (3) can be configured to receive a signal and can be coupled to an analog-to-digital converter. Another channel (4) can be electrically coupled to ground. In a particular example, the third channel (3) and fourth channel (4) can be coupled to a sensor that is applied to the patient. Using such a sensor, aspects of the electrical signal applied to the patient can be monitored. For example, the system can monitor amplitude of the signal, impedance in the patient's tissue, or other properties of the signal or patient's tissue and can automatically adjust the amplitude or waveforms of the signals applied to the patient through the first or second channels (1 or 2).

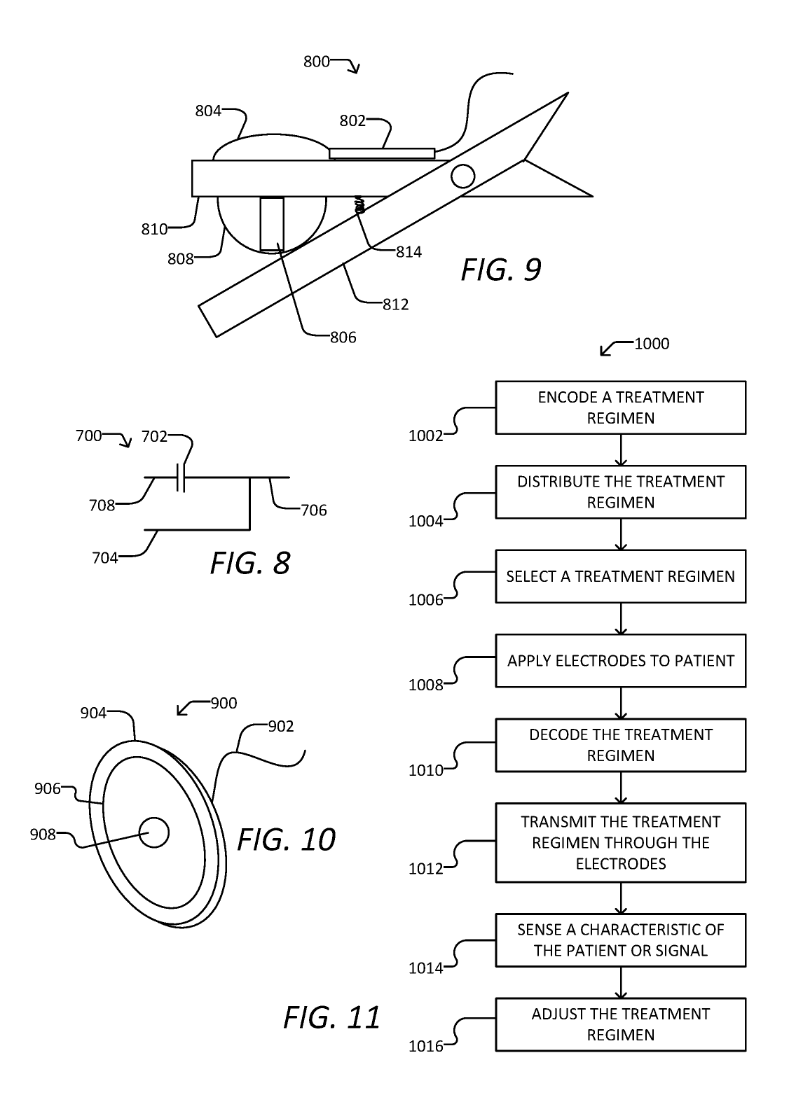

In a particular example illustrated in FIG. 8, a circuitry connected to one or more channels or leads can be circuitry 700 and that includes a power output 704 coupled directly to the channel 706. The power output 704 can be a DC output. The frequency signal 708 can be capacitively coupled through a capacitor 702 to the channel 706. The DC power can you range between 0 V and 5 V, such as between 0V and 3 V, or between 0.5 V and 2.0 V, or approximately 1.5 V.

Leads can be coupled to a patient, providing direct electrical contact or capacitively coupling to the patient. In an example, the leads can be connected to a clip or pad. For example, a clip 800 illustrated in FIG. 9 can be coupled to the patient, such as the patient's ear, finger, or fold of skin. A lead 802 be coupled with an electrical interface 804 of the clip 800. The interface 804 can be electrically coupled to an electrode 806 to contact the patient. The electrode 806 can directly contact the patient, providing direct electrical contact. In another example, an insulative coating can be applied over a surface of the electrode 806 to provide capacitive coupling. Alternatively, a capacitor can be located electrically between the interface 804 and the electrode 806 to provide capacitive coupling to the patient.

Optionally, a resilient pad 808 can be provided around the electrode 806. For example, the resilient pad 808 can be a foam or rubber pad to improve comfort for the patient. In a further example, the electrode 806 and resilient pad 808 can be combined utilizing electrically conductive resilient materials. In a particular example, the clip 800 includes an upper frame 810 to which the interface 804 and electrode 806 are secured and includes a lower frame 812 to provide a counter force to the upper frame 810 to secure the electrode to the patient. The clip 800 can also include a spring or resilient member 814 to draw the upper frame 810 and lower frame 812 together.

In another example illustrated in FIG. 10, a pad 900 can be connected to a lead 902. The pad 900 can include a support 904 to which gel or adhesive 906 can be applied. Optionally, the gel or adhesive 906 is conductive. The lead 902 is electrically coupled to electrode 908, which is coupled to patient either directly or through the gel or adhesive 906. For example, the electrode 908 can be directly electrically coupled to the patient through a conductive gel. In another example, the electrode 908 can be capacitively coupled to the patient through a nonconductive gel. Alternatively, the electrode can disrupt the outer layers of the skin. For example, the electrode can include a needle.

In an exemplary embodiment, a treatment regimen including a combination of therapeutic frequencies can be encoded into a file, such as an audio format file, which is distributed to potential users or patients. The files can be incorporated into a mobile application or can be accessed from an audio library. Patients can select a desired treatment regimen on a device and apply electrodes coupled to the device. The device can decode the encoded treatment frequencies and transmit those frequencies through the electrodes as an analog signal to treat a patient.

For example, as illustrated in FIG. 11, a method 1000 includes encoding a treatment regimen, as illustrated at 1002. In particular, the treatment regimen can include a set of treatment frequencies. In particular, the treatment frequencies can be two or more different frequencies encoded to be applied concurrently, for example, simultaneously. The treatment regimen can be encoded in a digital format, such as an audio file format. Exemplary audio file formats include a format compliant with a Moving Picture Experts Group (MPEG) standard (e.g., MPEG-1, MPEG-2, MPEG-4, or combinations thereof), AAC, ALAC, WAV, WMAL, a custom format, or the like, or a combination thereof.

As illustrated at 1004, the treatment regimen including the encoded therapeutic frequencies can be distributed to potential users or patients. In a particular example, utilizing an audio format, the treatment regimen can be distributed electronically, such as through an online marketplace or a music marketplace. Distribution can include providing a selected or purchased audio file to be stored on a user device, such as through downloading through a network. In an alternative example, distribution can include streaming a selected treatment regimen in an audio format to the user device. Such downloading and selecting can be performed over a global network (e.g., the Internet). Alternatively, an audio format file can be stored on the device and selected from the audio device. In another example, the encoded files can be associated with a mobile application that can be downloaded to the device, for example, from a mobile application marketplace.

As illustrated at 1006, when a patient is ready to be treated, the treatment regimen can be selected. For example, one or more treatment regimens can be stored on the device. The treatment regimens can be stored in a separate set of files, as part of an audio file library, such as a music library, or can be stored in association with a mobile application. The user can utilize user interfaces, such as touchscreens or buttons, to select the desired treatment regimen. Alternatively, in an example of a streamed treatment regimen, a user can access a listing through a network and facilitate streaming of the data associated with the treatment regimen and encoding the therapeutic frequencies to be sent to the device.

Electrodes can be applied to the patient or user and electrically coupled to the device, as illustrated at 1008. For example, one or more clips can be attached to the patient in a desired configuration to treat the selected tissue or region of tissue. In an example, the clip can be applied to an ear. In another example, a pad can be applied to the skin proximate to the tissue to be treated. Alternatively, the electrode can include a needle.

The device can decode the treatment regimen including the therapeutic frequencies from the associated file or streaming data, as illustrated at 1010. In particular, in the case of audio format file encoding the therapeutic frequencies, the device can decode the therapeutic frequencies using an associated audio codec. The decoded signal can be a digital signal, which is converted by a digital-to-analog converter and applied through an audio jack of the device to the electrodes coupled to the patient.

As illustrated at 1012, the analog electrical signal incorporating the therapeutic frequencies can be transmitted from the electrodes to the patient as electrical energy waves. The electrodes can electrically couple to the patient directly or can be capacitively coupled to the patient and provide the therapeutic frequencies to the tissue proximal to the electrodes. In an example, the therapeutic frequencies can be applied for duration of 15 seconds to 30 min. For example, the duration can be between 15 seconds and 20 minutes, such as between 30 seconds and 15 minutes, 1 minute and 15 minutes, 1 minute and 10 minutes, or 1 minute to 8 minutes.

Optionally, in the case of an additional sensor being attached to the patient, the device can sense aspects of the patient or electrical signal, as illustrated at 1014. In an example, the device can detect impedance within the patient's tissue. In a particular example, the device can automatically adjust aspects of the electrical signal applied patient, such as amplitude or a waveform, based on the sensed measurement, as illustrated at 1016.

The treatment regimen can be applied between 1 and 10 times per day, such as between 3 and 8 times.

In particular, the treatment can be applied to treat ailments of the skin, ailments associated with subcutaneous tissue, or ailments associated with nerve damage or nerve signal hindrance. For example, the treatment can be used for ailments of the skin, such as acne or rashes. In another example, the treatment can be applied to treat ailments associated with nerve damage or nerve signal hindrance, such as Bell's palsy. In particular, a combination of therapeutic frequencies can be applied concurrently to treat foreign bodies or structures.

### EXAMPLES

### Example 1

A patient is diagnosed with Bell's palsy, prescribed prednisone, and advised that the condition will start to improve in the next several months to a year or more, but perhaps would never recover completely.

The patient is introduced to a treatment regimen utilizing electric energy waves incorporating therapeutic frequencies. The patient attaches electrodes to the ears and applies the treatment regimen 8 times per day for a duration of 5 minutes each time using an application running on an IPad. The treatment regimen includes concurrently transmitting frequencies in a range of 70 Hz to 1 kHz.

The patient reports seeing the effects of the treatment regimen immediately and significant improvement in the condition within 6 days.

### Example 2

A patient has severe acne. Typical treatment techniques are differed, although daily cleaning of the affected area continues. The patient is introduced to a treatment regimen using electrical energy waves incorporating therapeutic frequencies. The patient attaches electrodes to the ears and applies the treatment regimen. Over the next several days the patient reports significant improvement and is continuing to use the technology.

In a first aspect, a method for treating a patient includes encoding a therapeutic frequency in a digital file to be stored on a device having an audio jack; applying electrodes connected to the device through the audio jack to the patient; and playing the digital file with the device, the therapeutic frequencies applied to the patient as electrical energy waves.

In an example of the first aspect, the digital file is encoded in a format selected from the group consisting of a Moving Picture Experts Group (MPEG) standard, AAC, ALAC, WAV, WMAL, a custom format, and a combination thereof.

In another example of the first aspect and the above examples, the method further includes selecting the digital file from a list of therapeutic digital files displayed on a user interface of the device prior to playing the digital file with the device.

In a further example of the first aspect and the above examples, playing the digital file includes decoding the digital file using an audio codec. For example, playing the digital file further includes converting a digital signal decoded using the audio codec to an analog signal incorporating the therapeutic frequencies.

In an additional example of the first aspect and the above examples, the therapeutic frequencies have a frequency in a range of 0.1 kHz to 2000 kHz.

In another example of the first aspect and the above examples, the therapeutic frequencies include a first frequency having a frequency range of 0.1 kHz to 2000 kHz and a second frequency having a frequency range of 0.1 kHz to 2000 kHz, the first and second frequencies to be applied to the concurrently.

In a further example of the first aspect and the above examples, playing the digital file includes applying the therapeutic frequencies for a duration of 15 second to 20 minutes. For example, the duration is in a range of 1 minute to 10 minutes.

In an additional example of the first aspect and the above examples, the electrodes are direct contact electrodes.

In another example of the first aspect and the above examples, the electrodes are capacitively in communication with the patient.

In a further example of the first aspect and the above examples, applying the electrodes to the patient includes electrically coupling an electrode to an ear of the patient.

In an additional example of the first aspect and the above examples, applying the electrodes to the patient includes electrically coupling an electrode to a finger of the patient.

In another example of the first aspect and the above examples, the audio jack is a single channel jack.

In a further example of the first aspect and the above examples, the audio jack is a multichannel jack. For example, the therapeutic frequencies applied to a first channel of the multichannel jack are an inverse of the therapeutic frequencies applied to the second channel of the multichannel jack. In another example, the therapeutic frequencies applied to a first channel of the multichannel jack have a different timing relative to the therapeutic frequencies applied to the second channel of the multichannel jack.

In an additional example of the first aspect and the above examples, the therapeutic frequencies are effective in treating Bell's palsy.

In another example of the first aspect and the above examples, the therapeutic frequencies are effective in treating acne.

In a second aspect, a method of treating a patient includes decoding a digital file encoding a set of therapeutic frequencies using an audio codec of a device, the device including an audio jack; applying electrodes to a patient, the electrodes electrically connected to the audio jack of the device; and transmitting an electrical energy wave signal corresponding to the therapeutic frequencies through the patient via the electrodes.

In an example of the second aspect, the digital file is encoded in a format selected from the group consisting of a Moving Picture Experts Group (MPEG) standard, AAC, ALAC, WAV, WMAL, a custom format, and a combination thereof.

In another example of the second aspect and the above examples, the method further includes selecting the digital file from a list of therapeutic digital files displayed on a user interface of the device prior to decoding the digital file with the device.

In a further example of the second aspect and the above examples, decoding the digital file further includes converting a digital signal decoded using the audio codec to an analog signal incorporating the therapeutic frequencies.

In an additional example of the second aspect and the above examples, the therapeutic frequencies have a frequency in a range of 0.1 kHz to 2000 kHz.

In another example of the second aspect and the above examples, the therapeutic frequencies include a first frequency having a frequency range of 0.1 kHz to 2000 kHz and a second frequency having a frequency range of 0.1 kHz to 2000 kHz, the first and second frequencies to be applied to the concurrently.

In a further example of the second aspect and the above examples, playing the audio file includes applying the therapeutic frequencies for a duration of 15 second to 20 minutes.

In an additional example of the second aspect and the above examples, the electrodes are direct contact electrodes.

In another example of the second aspect and the above examples, the electrodes are capacitively in communication with the patient.

In a further example of the second aspect and the above examples, applying the electrodes to the patient includes electrically coupling an electrode to an ear of the patient.

In an additional example of the second aspect and the above examples, applying the electrodes to the patient includes electrically coupling an electrode to a finger of the patient.

In another example of the second aspect and the above examples, the audio jack is a single channel jack.

In a further example of the second aspect and the above examples, the audio jack is a multichannel jack. For example, the therapeutic frequencies applied to a first channel of the multichannel jack are an inverse of the therapeutic frequencies applied to the second channel of the multichannel jack. In another example, the therapeutic frequencies applied to a first channel of the multichannel jack have a different timing relative to the therapeutic frequencies applied to the second channel of the multichannel jack.

In additional example of the second aspect and the above examples, the therapeutic frequencies are effective in treating Bell's palsy.

In another example of the second aspect and the above examples, the therapeutic frequencies are effective in treating acne.

In a third aspect, a method of treating a patient includes selecting a treatment regimen from a plurality of treatment regimens through a user interface of a device, the device including an analog output and the user interface, each treatment regimen of the plurality of treatment regimens associated with a file encoding a digital treatment signal; decoding the file using the device to produce the digital treatment signal; converting the digital signal to an analog treatment signal; and applying the analog treatment signal as an electric energy wave signal through the analog output and to a patient using an electrode in electrical communication with the analog output and the patient.

In a fourth aspect, a method of providing therapeutic regiments includes encoding a plurality of treatment regimens each in a treatment digital file, a treatment regimen of the plurality including a plurality of concurrent frequency signals; and distributing the plurality of treatment regimens to a file marketplace.

In an example of the fourth aspect, the method further includes accessing the file marketplace to download the treatment digital file. For example, accessing includes accessing through a global network. In another example, accessing includes accessing with a computational device and storing the treatment digital file on the computational device. In additional example, accessing includes accessing with a mobile device and storing the treatment digital file on the mobile device. In a further example, accessing includes streaming the digital file to a mobile device.

In another example of the fourth aspect and the above methods, the method further includes decoding the digital file to provide an analog treatment signal and applying the analog treatment signal to a user as an electrical energy wave. For example, applying the analog treatment signal includes directly electrically applying the analog treatment signal to the user. In another example, applying the analog treatment signal includes capacitively applying the analog treatment signal to the user.

In a fifth aspect, an apparatus includes a processor; a memory storage in communication with the processor; a power supply; an interface in communication with the processor; and a digital-to-analog converter in communication with the processor and a jack having an audio configuration; and a set of electrodes in electrical communication with the jack; wherein the processor is to retrieve an encoded treatment regimen from the memory storage in response to selection of the treatment regimen via the interface, decode the treatment regimen to a digital treatment signal, convert the digital treatment signal to an analog treatment signal using the digital-to-analog converter, and apply the analog treatment signal as electrical energy waves to the electrodes.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive- or and not to an exclusive- or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A method of providing therapeutic regimens, the method comprising:
encoding a plurality of treatment regimens each in a treatment digital file, a treatment regimen of the plurality including a plurality of simultaneous frequency signals; and
distributing the plurality of treatment regimens to a file marketplace.

2. The method of claim 1, further comprising accessing the file marketplace to download the treatment digital file.

3. The method of claim 2, wherein accessing includes accessing through a global network.

4. The method of claim 2, wherein accessing includes accessing with a computational device and storing the treatment digital file on the computational device.

5. The method of claim 2, wherein accessing includes accessing with a mobile device and storing the treatment digital file on the mobile device.

6. The method of claim 2, wherein accessing includes streaming the digital file to a mobile device.

7. The method of claim 2, further comprising decoding the digital file to provide an analog treatment signal and applying the analog treatment signal to a user as an electrical energy wave signal.

8. The method of claim 7, wherein applying the analog treatment signal includes directly electrically applying the analog treatment signal to the user.

9. The method of claim 7, wherein applying the analog treatment signal includes capacitively applying the analog treatment signal to the user.

10. A method for treating a patient, the method comprising:
encoding therapeutic frequencies in a digital file to be stored on a device having an audio jack;
applying electrodes connected to the device through the audio jack to the patient; and
playing the digital file with the device, the therapeutic frequencies applied to the patient as electrical energy waves.

11. The method of claim 10, wherein the digital file is encoded in a format selected from the group consisting of a Moving Picture Experts Group (MPEG) standard, AAC, ALAC, WAV, WMAL, and a combination thereof.

12. The method of claim 10, further comprising selecting the digital file from a list of therapeutic digital files displayed on a user interface of the device prior to playing the digital file with the device.

13. The method of claim 10, wherein playing the digital file includes decoding the digital file using an audio codec.

14. The method of claim 13, wherein playing the digital file further includes converting a digital signal decoded using the audio codec to an analog signal incorporating the therapeutic frequencies.

15. The method of claim 10, wherein the therapeutic frequencies have a frequency in a range of 0.1 kHz to 2000 kHz.

16. The method of claim 10, wherein the therapeutic frequencies include a first frequency having a frequency range of 0.1 kHz to 2000 kHz and a second frequency having a frequency range of 0.1 kHz to 2000 kHz, the first and second frequencies to be applied to the concurrently.

17. The method of claim 10, wherein playing the digital file includes applying the therapeutic frequencies for a duration of 15 second to 20 minutes.

18. The method of claim 17, wherein the duration is in a range of 1 minute to 10 minutes.

19. The method of claim 10, wherein the electrodes are direct contact electrodes.

20. The method of claim 10, wherein the electrodes are capacitively in communication with the patient.

21.-41. (canceled)
